(19) United States
(12) Patent Application Publication
(10) Pub. No.: US 2015/0025628 A1
(43) Pub. Date: Jan. 22, 2015

(54) Apparatus and Methods for Realistically Simulating the Human Nipple-Areolar Complex

(71) Applicant: Beveridge Marketing LLC, Potomac, MD (US)

(72) Inventor: Lucy Ruwitch Langer, Portland, OR (US)

(73) Assignee: Beveridge Marketing LLC, Potomac, MD (US)

(21) Appl. No.: 13/947,274

(22) Filed: Jul. 22, 2013

(51) Int. Cl. A61F 2/12

(52) U.S. Cl. CPC A61F 2/12
USPC 623/8

(57) ABSTRACT

An apparatus and method are disclosed for realistically simulating the appearance of a nipple-areolar complex. The apparatus may include a first flat surface having a non-toxic self-adhesive material and a second flat surface comprising a photographic image of a female human nipple complex. The photographic image may be a realistic depiction of a human nipple-areolar complex, having a first colored area resembling a human areola, and a second colored area formed within the first area, resembling a human teat.

### TECHNICAL FIELD

The invention is related generally to decals, adhesives, and temporary tattoos depicting realistic anatomical body parts, and in particular, to printed adhesive substrates and water-soluble tattoos realistically depicting a human nipple-areolar complex for use by mastectomy patients.

### BACKGROUND OF THE INVENTION

Each year, hundreds of thousands of women are diagnosed with breast cancer. For some women, a mastectomy is their only option for treatment. Mastectomies result in the partial or complete removal of the breast, and frequently, the nipple-areolar complex (“NAC”). This loss of the NAC often negatively impacts a woman's self-confidence and psychological well-being.

Several cosmetic and reconstructive options for improving the appearance of mastectomy patients are known, including breast and nipple reconstruction surgery, breast and nipple prostheses, and ink tattoos depicting nipples. However, each of these options carry significant drawbacks.

For example, the “simulated” NAC created as a result of breast and nipple reconstruction surgery, breast and nipple prostheses, or ink tattoos does not recreate the appearance of a natural NAC. Nor do these options recreate the particular patent's original NAC. Patients that undergo reconstructive surgery, for example, frequently complain that a reconstructed NAC does not appear realistic or similar to their original nipple, because reconstructive surgery typically involves recreating a NAC out of tissue grafted from different parts of the body. Likewise, NAC prostheses typically fail to realistically simulate the appearance of the patent's original NAC in terms of texture, color, shape, and size. Similarly, patients complain about the appearance of tattoos (permanent or temporary), because temporary tattoos generally depict low resolution caricaturized images that easily fade, rub-off, or deteriorate in image quality.

In addition to being unrealistic, these options do not offer patients the flexibility to inconspicuously or seamlessly wear their NAC. Reconstructed NACs or prosthetic NACs, for example, may embarrassingly protrude through a woman's apparel. Additionally, users of prosthetic NACs may be embarrassed about changing or removing them in front of others. On the other hand, because tattoos are not easily removable, they do not allow a user to change the NAC image tattooed on their breast, thereby preventing them from sampling different NAC images.

### SUMMARY OF THE INVENTION

One or more of the disadvantages outlined above can be addressed by implementing a self-adhesive apparatus having a photographic image of a female human NAC. Embodiments of the present invention include an apparatus having a first flat surface comprising a non-toxic self-adhesive material that is easily applied to and removed from a human breast area. The apparatus may further have a second flat surface depicting a photographic image of a female human NAC, including a first colored area resembling a human areola, and a second colored area formed within the first area, resembling a teat. The colors, shapes and sizes of the nipple-areolar image may be varied.

By imprinting a realistic image of a NAC onto a readily removable self-adhesive material, mastectomy patients may wear different shapes, colors, and sizes of nipple appearances. Imprinting a realistic or photographic image of a NAC onto a readily removable self-adhesive material also gives mastectomy patients the freedom to inconspicuously remove or change them.

### BRIEF DESCRIPTION OF THE DRAWINGS

FIGS. 1A and 1B depict an apparatus according to one embodiment of the invention.

FIGS. 2A and 2B depict embodiments of the invention that include image enhancements to the NAC.

FIGS. 3A and 3B depict embodiments of the invention that include commercial art, graphics, and/or promotional materials.

FIG. 4 depicts one embodiment of the invention wherein the NAC is lenticular.

FIG. 5 depicts a three-dimensional apparatus according to one embodiment of the invention.

FIG. 6 depicts one embodiment of the invention wherein the apparatus is applied like a temporary tattoo.

FIG. 7 depicts the placement location of the apparatus according to one embodiment of the invention on the human body.

### DETAILED DESCRIPTION OF PREFERRED EMBODIMENTS

Embodiments of the present invention include articles and methods for realistically depicting the human nipple-areolar complex.

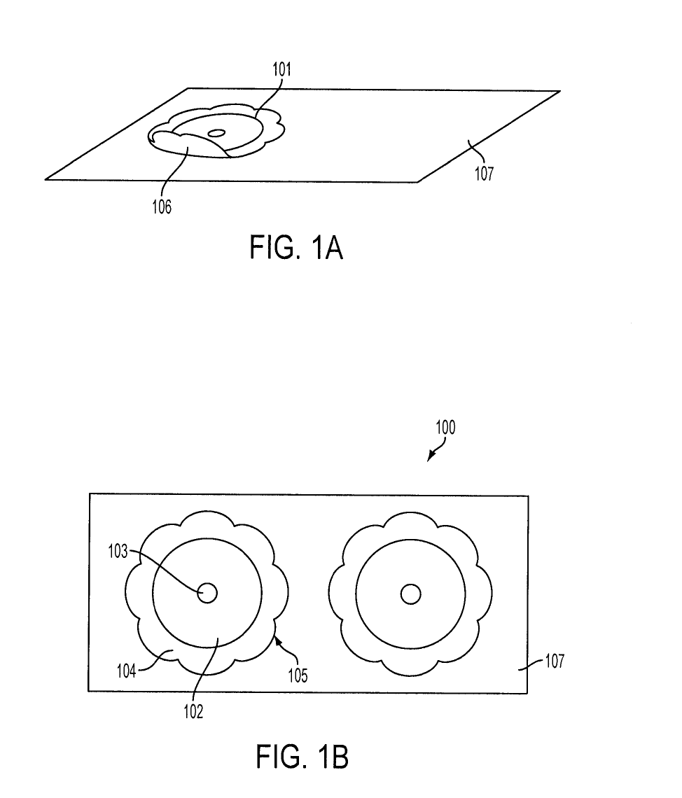

FIG. 1A is a perspective view of the NAC apparatus 100 according to one embodiment of the invention. Apparatus 100 may realistically simulate a female's NAC by depicting a life-like image of a woman's NAC. In preferred embodiments, the realistic image is a photograph that captures the intricacies and details of a female's NAC. Apparatus 100 may be made of a substantially transparent, non-woven medical grade tape, coated with a layer of adhesive, allowing women to seamlessly and inconspicuously remove the apparatus. Apparatus 100 may also include a border area around the image of the NAC, and have scalloped edges to help the apparatus 100 adhere to a user's uneven breast region.

The realistic image may be depicted on a first surface 101 of apparatus 100. In one aspect of the invention, the image of the NAC is a realistic and life-like depiction a woman's NAC. In comparison to reconstructed breasts, prostheses, or tattoos, embodiments of the invention use images that capture and simulate the life-like details and intricacies of a woman's NAC. For example, the NAC images may capture variations of skin tone or nipple pigmentation across a woman's NAC, including for example, freckles or birthmarks. As another example, the images may capture skin texture. Skin texture may be depicted in the images, for example, by depicting fine surface lines, wrinkles, dryness, or cracks in the areola or nipple. In yet another example of life-like intricacies, the images may capture skin irregularities such as skin tags, moles, or lesions. Other examples of realistic details include capturing asymmetric or irregular areolar and nipple shapes. Images of asymmetric or irregular areolar and nipple shapes may enhance the realism of the image for patients who, for example, have naturally ovular or lop-sided NACs. By using images of real-life NACs, embodiments of the invention capture features such as skin tone, skin texture, skin irregularities, and NAC shapes, that reconstructed breasts, prostheses, and tattoos fail to realistically portray.

According to one aspect of the invention, the image of the NAC may be a two-dimensional image based on a woman's actual, real-life NAC. For example, the image may be a photograph of a woman's real-life NAC, an illustration based on a woman's real-life NAC, or a computer rendering of a woman's NAC. In a preferred embodiment, the image is a photograph of a woman's NAC that captures the life-like and intricate features discussed above, i.e., skin tone, skin texture, skin irregularities, and NAC shapes. These features may be captured using cameras and printers with high-resolution and zoom capabilities. According to some embodiments, the first surface 101 is also two-dimensional (i.e., flat). In other embodiments (described in more detail below), the first surface 101 may be layered to provide depth, and the two-dimensional image may be printed on the layered first surface 101 to enhance the image's realism.

Photographs may depict the user's own NAC (e.g., a patient taking a photograph of her own NAC before undergoing a mastectomy), or someone else's NAC. One advantage to using images of someone else's NAC, is that users have the ability to preview what their appearance would be like with various different NAC shapes, colors, and sizes. For example, a user may “try on” a NAC apparatus 100 depicting an image of a NAC that is darker or larger than their natural NAC. This ability is especially useful for patients considering a permanent ink tattoo of a NAC, and are unsure of what particular shape, color, or size they would like their tattoo to be. With embodiments of the invention, a patient can effectively preview how a permanent ink tattoo with a similar shape, color, and size, would appear. Users may vary the size, shape, or color of the NAC image by finding an image of someone else with the desired size, shape, or color characteristics. According to other embodiments of the invention, the size, shape, or color of the NAC image may be varied by manipulating the image with photo-editing software well-known in the art, such as Adobe Photoshop®.

In one aspect of the invention, the life-like quality of the images may be further enhanced by including shading or lighting on or around the NAC. For example, in a photographic image of a NAC, the NAC may depict shading below the nipple, indicating a light source from above, casting a shadow below the nipple. In another aspect of the invention, the images may be coated with a glossy or reflective finish to simulate the reflectiveness of skin in natural light.

FIG. 1B illustrates apparatus 100 according to one embodiment of the invention. Apparatus 100 shows an image of a woman's NAC depicted on the first surface 101, comprising a first colored area resembling an areola 102 and a second colored area resembling a human teat 103. As described above, when viewed together, the first colored area 102 and second colored area 103 combine to form a realistic and life-like depiction a woman's NAC. That is, the areola depicted in the first colored area 102 captures and simulates the life-like details and intricacies of a woman's areola. Likewise, the teat depicted in the second colored area 103 captures and simulates the life-like details and intricacies of a woman's teat.

According to some embodiments of the invention, the first colored area 102 and second colored area 103 may exhibit varying degrees of transparency. For example, in some embodiments of the invention, the first colored area 102 and second colored area 103 may be tinted with a user's skin tone and partially transparent. In these embodiments, the partial transparency of the colored areas allows the user's underlying breast region to be partially visible, causing apparatus 100 to blend in with the pigmentation of the skin, thereby enhancing the realism of apparatus 100.

According to other embodiments of the invention, the NAC image may be altered or manipulated to specifically conceal scars on the patient's breast region. For example, apparatus 100 may depict a particular skin tone on a targeted area of the areola 102, or border 104 (described below) to conceal a scar underneath that area of the apparatus. In yet further embodiments of the invention, a waterproof color marker (not pictured) may be provided with the apparatus 100. The waterproof color marker may be used to mark up the area depending on the angle or location she chooses to place the apparatus on.

In one aspect of the invention, the image of the NAC, including the first colored area 102 and second colored area 103, may be printed on apparatus 100 using printing materials and printing techniques well-known in the art. For example, apparatus 100 may be a substrate made of thin materials adapted for printing, such as a cloth, paper, silicone, plastic, or metal. In a preferred embodiment of the invention, the substrate may be a 3M™ non-woven substantially transparent medical grade cloth tape. Printing techniques for imprinting the image onto apparatus 100 may include flexographic printers, letterpress printers, screen printers, gravure printers, offset lithographic printers, or digital printers. In a preferred embodiment of the invention, the image is printed using a digital flexographic printer. These printing techniques may also be used to print other types images onto apparatus 100, such as commercial art (described below).

According to some embodiments of the invention, the first surface 101 may further be coated with different fragrances or flavors. Micro-fragrance coatings may be applied to the first surface 101 using techniques well-known in the art. For example, a micro-coating fragrance may be applied through the process of micro-encapsulation, creating micro-encapsulated bubbles on the first surface. The micro-encapsulated aroma bubbles may then be broken through contact such as scratching or rubbing, releasing the odor of the fragrance. Likewise, in embodiments where the first surface has been coated with flavors, coatings are applied to the first surface using techniques well-known in the art. For example, sweet or savory notes may be imported onto a first surface 101 through micro-encapsulation, and emulsification techniques. Micro-encapsules, may comprise small capsules of active agents or core materials and ingredients surrounded by a shell or coating, that is released upon a physical, chemical, or physico-chemical reaction, such as changes in composition, acidity, temperature, or light. Upon a releasing trigger, the micro-encapsules are released, delivering a sensation of taste to the user.

The NAC apparatus 100 may further comprise a border 104, and trimmed edges 105. The border 104 and trimmed edges 105 provide extra surface area for the NAC apparatus 100 to adhere to the user's breast region.

In one aspect of the invention, border 104 and edges 105 may exhibit varying degrees of transparency. In contrast to the first colored area 102 and second colored area 103, which may be tinted with color, border 104 and trimmed edges 105 may be clear and wholly transparent. Thus, when the NAC apparatus is applied to a user's breast region, the border 104 and trimmed edges 105 inconspicuously blend in within the user's skin, unnoticeable to other viewers.

In another aspect of the invention, the trimmed edges 105 of the substrate may be trimmed into different shapes to help apparatus 100 adhere to the uneven surface of a user's skin. For example, in one embodiment of the invention, the edges of apparatus 100 may be scalloped, as depicted in FIGS. 1A and 1B. In other embodiments, the edges may be trimmed into other geometric-shaped edges, such as rounded, ovular, elliptical, foiled (e.g., trefoiled, quatrefoiled, cinquefoiled), or curvilinear polygoned, to improve the grip between the substrate and the user's skin.

Apparatus 100 further comprises a second surface 106. The second surface 106 comprises an adhesive coating that allows the NAC apparatus 100 to adhere to a user's breast region. The adhesive material may be pressure-sensitive, such that the adhesive material bonds the user's skin to the substrate when the user applies the NAC apparatus to their breast. According to some embodiments of the invention, the self-adhesive material may be a pressure-sensitive adhesive, such as the 3M™ Pressure Sensitive Adhesive, or 3M™ Medical Grade Instant and Light Cure Adhesives. The type of substrate and adhesive material chosen may vary depending on the characteristics of the user. For example, the type of substrate and adhesive may depend on a user's skin surface characteristics, the activity level of the user, and anticipated wear time. In some embodiments of the invention, the substrate and adhesive may be waterproof or water-repellant, allowing users to swim, shower, and otherwise expose apparatus 100 to water and other liquids. In preferred embodiments of the invention, the adhesive and substrate is selected so that the apparatus 100 stays bonded to the user's skin for a minimum of two days.

In one aspect of the invention, the second surface 106 may include a coating of medicine or therapeutic ointment. For example, the second surface 106 may include a coating of therapeutic ointment, such as Vitamin E to assist with a mastectomy patient's skin regeneration or scar tissue. In other embodiments, the second surface 106 may include a coating of medicine. For example, the second surface 106 may include topical corticosteroids, anesthetic ointments, or antihistamine creams to reduce symptoms of itching and tenderness.

Apparatus 100 may further comprise a release liner 107. The release liner 107 is a carrier material coated on its surface with a release agent that provides a release effect against most types of adhesive or mastic material. In one aspect of the invention, the second surface 106 is attached to a release liner 107 by the adhesive material described above. As FIG. 1A illustrates, apparatus 100 may be detached from the release liner 107 by peeling on the corner of the second surface 102.

According to some embodiments of the invention, the release liner 107 may be made of liner materials well-known in the art, such as a paper-, polyester-, plastic-, or high-density-polyethylene-based material, that carries a release agent for transporting adhesive materials. In a preferred embodiment of the invention, the release liner 107 is a paper-based liner made of 3M™ Densified Kraft Secondary Liner 4994 paper. According to some embodiments of the invention, the release agent may be a silicone-based release agent, such as the 3M™ Silicone Lubricant.

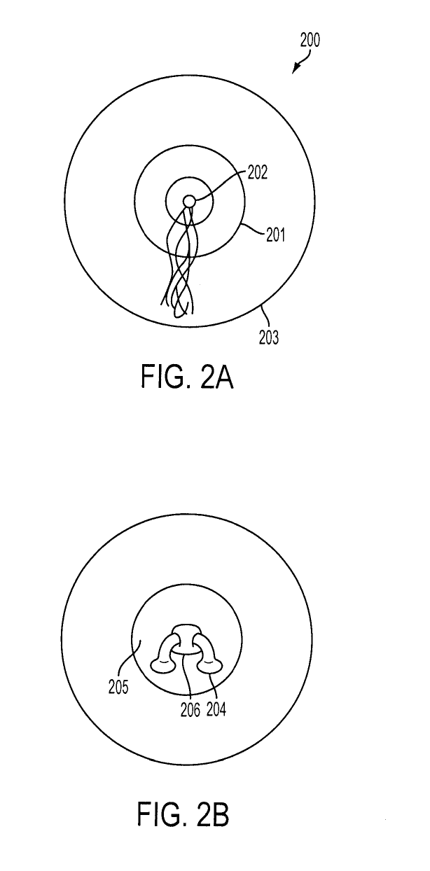

FIGS. 2A and 2B depict a NAC apparatus 200 according to some embodiments of the invention that include NAC image enhancements for recreational or entertainment use. NAC apparatus 200 shows an areola image 201, an accessory image 202, and a border with trimmed edges 203. The areola image 201 may be a realistic image of the areola as described above. An accessory image 202 may be an image of a nipple accessory that partially or wholly covers the teat, such as a nipple tassel. The border with trimmed edges 203 provides additional surface area to improve the adherence of the NAC apparatus 200 to the breast, as described above. In one aspect of the recreational NAC apparatus 200, the image of the accessory extends beyond the teat and areola and into the border 203. For example, to convey the realism of the nipple tassel, the tassel threads may be depicted as hanging below the areola as depicted in the Figure. As described above, in one aspect of the invention, the image of the accessory depicts several details and intricacies that create a life-like and realistic appearance. For example, the threads of the tassels, and their knots and bunches, may be depicted in the image. As described in more detail below, the realism of the tassels may be further enhanced by using lenticular or holographic printing methods to create the visual effect of movement and space.

FIG. 2B illustrates a NAC apparatus 200 comprising an areola image 205, an accessory image 204, an image of a teat 206, and a border with trimmed edges 207. The areola image 205 and teat image 206 may be realistic images of the areola and teat as described above. An accessory image 204 may be an image of a nipple accessory that partially covers the teat, such as a nipple ring. The border with trimmed edges 207 provides additional surface area to improve the adherence of the NAC apparatus 200 to the breast, as described above. In one aspect of the recreational NAC apparatus 200, the image of the accessory enters the teat or areola. For example, to convey the realism of the nipple ring, the ring may be depicted as piercing through the teat or areola as depicted in the Figure. As described above, in one aspect of the invention, the image of the accessory depicts several details and intricacies that create a life-like and realistic appearance. As with the NAC images described above, the details and intricacies of the accessories may be captured to provide a life-like and realistic feel to the image. For example, the image may depict the reflective properties commonly observed on metallic surfaces such as rings.

Other types of accessories, such as pasties, shields, glitter, sparkles, or jewelry for example, may be included in recreational or entertainment NAC apparatuses.

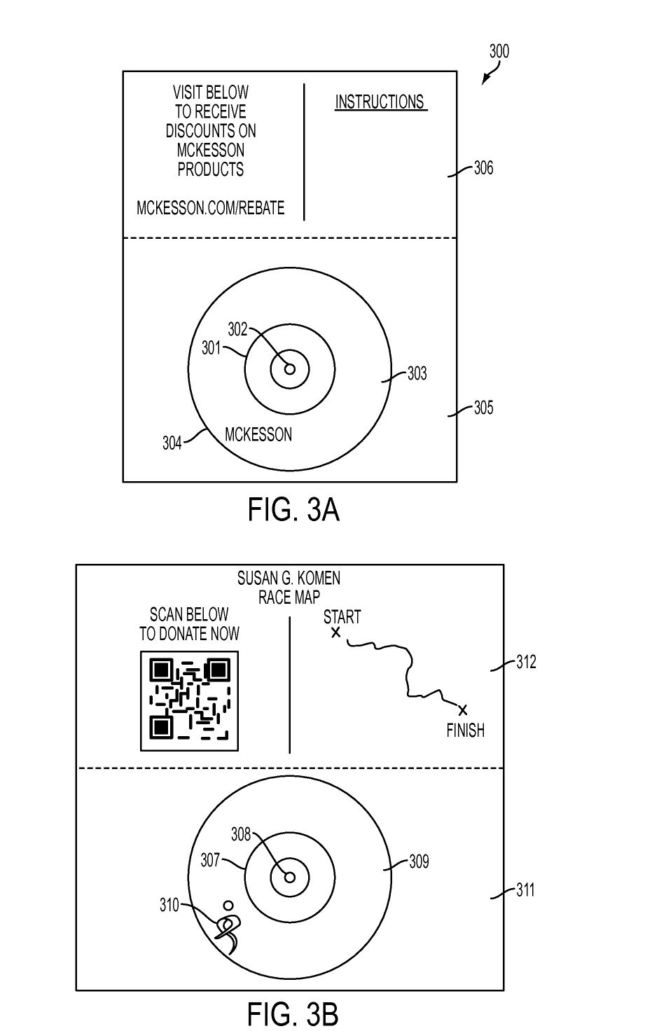

FIGS. 3A and 3B depict NAC apparatuses 300 according to some embodiments of the invention that include commercial art and/or promotional materials. FIG. 3A illustrates a NAC apparatus 300 comprising an image of an areola 301, a teat 302, a border and trimmed edges 303, a commercial illustration 304, and a release liner 305. Some embodiments of the invention may further include promotional materials 306. The areola image 301 and teat image 302 may be realistic images of the areola and teat as described above. The border with trimmed edges 303 provides additional surface area to improve the adherence of the NAC apparatus 300 to the breast, as described above. The release liner 305 is a carrier material coated on its surface with a release agent as described above. Promotional materials 306 provide promotions and/or content that may be related to the commercial illustration 304. In one aspect of the NAC apparatus 300, the commercial art may be the name, logo, or trademark of a business entity or institution, such as hospital or medical practice group. For example, the Figure depicts the name of a popular medical product provider, McKesson. According to some embodiments, the commercial art may appear inconspicuously on the border 304 as a faint imprint, such as a water mark. Such a watermark may exhibit varying degrees of transparency. According to some embodiments of the NAC apparatus 300, the apparatus may include promotional materials 306. Promotional materials 306 may provide promotions for the user to redeem, such as discounts or coupons to products or services related to the company, business entity, or institution depicted in the commercial illustration 304. For example, the promotional materials may include a link to a website that allows the user to redeem a coupon or rebate for McKesson products. Promotional materials 306 may also depict content, such as instructions for applying the apparatus, or information about the company, business entity, or institution depicted in the commercial illustration.

FIG. 3B illustrates a NAC apparatus 300 comprising an image of an areola 307, a teat 308, a border and trimmed edges 309, a commercial illustration 310, and a release liner 311. Some embodiments of the invention may further include promotional materials 312. The areola image 307 and teat image 308 may be realistic images of the areola and teat as described above. The border with trimmed edges 309 provides additional surface area to improve the adherence of the NAC apparatus 300 to the breast, as described above. The release liner 311 is a carrier material coated on its surface with a release agent as described above. Promotional materials 312 provides promotions and/or content that may be related to the commercial illustration 310. In one aspect of the NAC apparatus 300, the commercial art may be the name, logo, or trademark of a business entity or institution, such as hospital or medical practice group. For example, the commercial art depicted in the Figure is the Susan G. Komen® ribbon, typically associated with breast cancer initiatives. According to some embodiments of the NAC apparatus 300, the apparatus may include promotional materials 312. Promotional materials 312 may provide promotions for the user to redeem, such as discounts or coupons to products or services related to the company, business entity, or institution depicted in the commercial illustration 310. For example, the promotional materials may include a QR code that a user may scan to allow the user to donate to the Susan G. Komen foundation. Promotional materials 312 may also depict content, such as a map for a breast cancer awareness race.

According to other embodiments of the invention, the commercial art may depict cartoons or other recreational images. For example, the commercial art may be stock images of puppies, smiley faces, or butterflies, and the like. Thus, the NAC apparatus 300 may depict a life-like and detailed image of the areola 307 and teat 308, on the one hand, while simultaneously depicting a cartoon or recreational image 310 on the other.

According to other embodiments of the invention, the commercial art may depict a graphical design resembling a tattoo (not pictured). For example, the commercial art may depict popular tattoo designs, such as butterflies, zodiac symbols, tribal patterns, and the like, on the border 304 of the NAC apparatus 300. As with the areola 307 and teat 308 images described above, the tattoo images may capture life-like details and intricacies enhancing the realism of the tattoo. For example, the images may capture the fading or bleeding of inks that are commonly observed with tattoos. In one aspect of the invention, the areola, teat, or commercial art images may be printed onto the substrate of the NAC apparatus 300 using ink with special reflective and/or optical properties. For example, the areola, teat, or commercial art images may be printed with florescent, glow-in-the-dark inks, or metallic-colored inks.

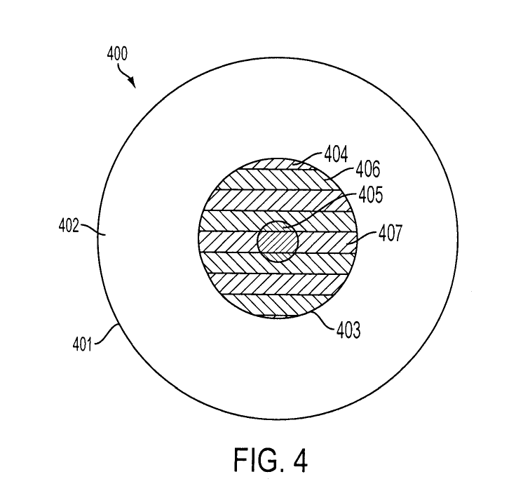

FIG. 4 is an aerial view of a lenticular NAC apparatus 400 according to one embodiment of the invention. The lenticular NAC apparatus 400 may comprise a substrate 401 as described above, a lenticular image of the NAC 403, and a border 402 as described above. The lenticular image 403 creates the effect of motion or depth when viewed at different angles.

The lenticular NAC image 403 is created by employing lenticular printing methods onto the substrate 401. Lenticular printing is a multi-step process that typically includes interlacing and combining at least two images with a lenticular lens. Thus, the lenticular image 403 comprises at least two images of a woman's NAC, which are depicted in FIG. 4 as an image of a first areola 404, an image of a first teat 405, an image of a second areola 406, and an image of a second teat 407. Each image may be a realistic image of a woman's NAC as described above. Once the images are interlaced and combined using methods well-known in the art, the lenticular image 403 creates the effect of motion or depth when viewed at different angles. Although FIG. 4 depicts two images of women NACs, embodiments of the invention may comprise any number of women's NAC images.

The two-dimensional images of a woman's NAC may be enhanced with other three-dimensional visual and motion effects well-known in the art. For example, the image may be a flat-panel spatially multiplexed parallax, i.e., autosteroscopic, display, such as a hologram. Holograms may be imprinted onto the substrate using holographic printers, substrates, and printing techniques well-known in the art. According to one embodiment, the image may be imprinted on a thin sheet of plastic or metallic film, such as aluminum or steel, by embossing the image in a detailed pixelated relief. When placed on a horizontal surface and illuminated with an appropriate light source, the irregularities in the surface produce a three-dimensional, full-parallax, color or monochromatic image that appears to float above the surface, by scattering the reflected light and diffracting it into its constituent color wavelengths. The reflected waves interfere with each other so as to give a vivid, realistic three-dimensional portrayal of perspective and parallax.

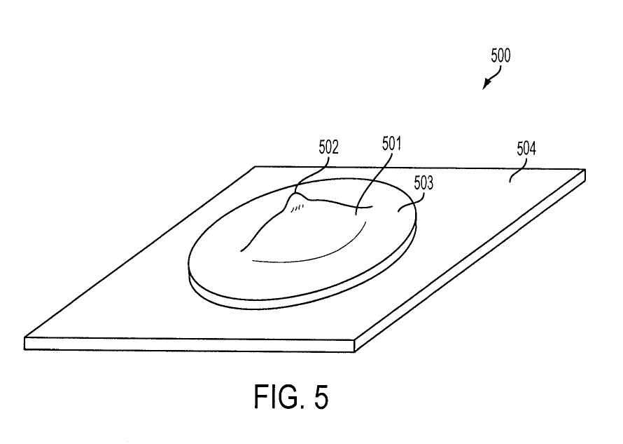

FIG. 5 is a perspective view of a three-dimensional NAC apparatus 500 according to one embodiment of the invention. The substrate of apparatus 500 is three-dimensional, i.e., not flat, to provide additional depth to the image of the NAC. For example, the surface of apparatus 500 may be slightly elevated in different regions, in the same way that a real human NAC conveys depth and volume. For example, the surface area where an image of areola would be depicted may be slightly elevated just as the surface of a real-life areola, by using an additional layers of substrate material on that area.

Apparatus 500 comprises an image of an areola 501, a teat 502, a border and trimmed edges 503, and a release liner 504. The areola image 501 and teat image 502 may be realistic images of the areola and teat as described above. The border with trimmed edges 503 provides additional surface area to improve the adherence of the NAC apparatus 500 to the breast, as described above. The release liner 504 is a carrier material coated on its surface with a release agent as described above. The surface of the apparatus is slightly elevated in different regions to provide depth to the images of the areola 501 and teat 502. For example, the surface area of the image of the areola 501 may be slightly elevated just as the surface of a real-life areola, by using an additional layers of substrate material on that area. Similarly the image of the teat 502 may be elevated by using additional layers of substrate. According to other embodiments of the invention, other life-like details and intricacies of the skin may be enhanced three-dimensionally by adding additional substrate layering. For example, an image depicting a skin tag or mole may be elevated in the same manner they would be in real-life with additional substrate layers.

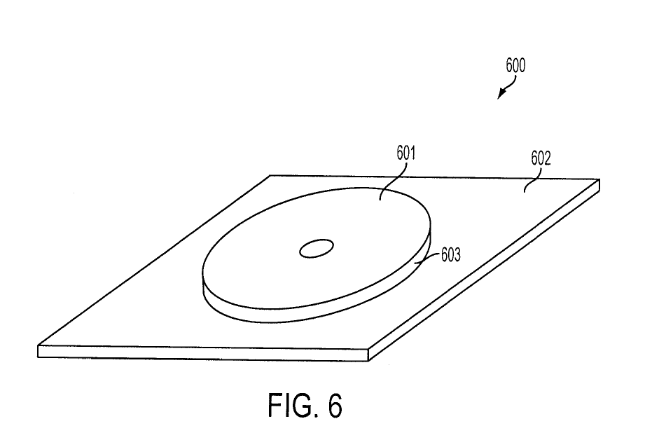

FIG. 6 illustrates a NAC apparatus 600 according to temporary tattoo embodiments of the invention. Apparatus 600 comprises an image of a woman's NAC 601 and a release liner 602. According to some embodiments, apparatus 600 may further include a transfer film 603. The image of the NAC may be a realistic image of a woman's NAC as described above with respect to self-adhesive substrates. The release liner 602 is a carrier material coated on its surface with a release agent as described above. Similarly to the printed adhesive substrates above, apparatus 600 is applied by printing the image of a woman's NAC to a transfer film 603 or release liner 602 using methods well-known in the art. For example, the image may be printed onto the transfer film 603 or release liner 602 using flexographic printers, letterpress printers, screen printers, gravure printers, offset lithographic printers, or digital printers. The image is transferred from the transfer film 603 or release liner 602 to a user's skin by dampening the skin, and firmly holding the transfer film 603 or release liner 602 against the skin. After a short duration of time, typically one to three minutes, the transfer film 603 or release liner 602 will soften, and the release liner 602 can be peeled away, leaving the printed image intact on the user's skin. As described above, apparatus 600 may also include images for recreational or entertainment use, commercial art, promotional items, and lenticular images.

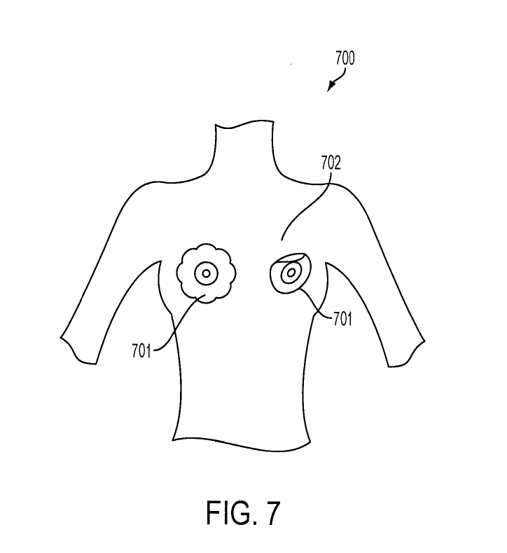

FIG. 7 illustrates a user applying a NAC apparatus 700 according to one embodiment of the invention. FIG. 7 depicts a user applying apparatus 701 to a breast region 702. According to embodiments of the invention where the NAC image is printed on a printed adhesive substrate, a user may apply apparatus 701 by peeling the substrate off a release liner, and gently pressing the substrate against the user's breast region 702. As discussed above, the user may smooth their hands over the trimmed edges of the substrate to increase adherence between the substrate and curvilinear surface of the user's skin. A user may easily remove the substrate by firmly gripping a corner of the substrate and peeling away from the skin. In temporary tattoo embodiments of the invention, the user may transfer the image of the woman's NAC from the transfer film or release liner by dampening the skin, and firmly holding the transfer film or release liner against the skin. The image may then be removed by washing the ink and skin with a gentle cleanser, such as baby oil or soap.

Those of ordinary skill in the art will appreciate that the embodiments of the invention are not limited to depicting images of the NAC, but may broadly apply to any other anatomical body part. For example, instead of depicting an image of a female's NAC, the apparatus may depict a toe, toenail, finger, fingernail, ear, earlobe, or elbow. Moreover, the invention may generally be applied to any skin surface region. According to some embodiments, the invention may be used to conceal damaged skin surface regions, or skin irregularities. For example, in one embodiment, burn victims with burn scars on a region of their skin, may print a realistic image of unscarred skin on a printed adhesive substrate. The printed adhesive substrate may inconspicuously conceal the damaged skin surface by replicating the skin tone and texture of the victim's skin. In another embodiment, a person may conceal an unsightly blemish or skin discoloration, by covering the skin surface region with a realistic image of the user's skin (e.g., by matching the user's skin tone, texture, and similar life-like intricacies) on a printed adhesive substrate.

What is claimed is:

1. An apparatus for realistically simulating the appearance of a nipple-areolar complex, the apparatus comprising:
an adhesive substrate comprising non-woven medical-grade tape for depicting a realistic image of a nipple-areolar complex and trimmed edges for improving adherence to uneven surfaces, said substrate comprising a first flat surface and a second flat surface;
wherein said first flat surface comprises a photographic image of a female human nipple complex, the photographic image comprising:
a first colored area resembling a human areola, and
a second colored area formed within the first area, wherein the second area resembles a human teat; and
wherein said second flat surface comprises a non-toxic self-adhesive material for adhering said apparatus to human skin.

2. The apparatus of claim 1, wherein the photographic image depicts the user's own nipple complex.

3. The apparatus of claim 1, wherein the photographic image further comprises computer-generated graphics.

4. The apparatus of claim 1, wherein the photographic image further comprises illustrations.

5. The apparatus of claim 1, wherein the apparatus' edges are scalloped, rounded, foiled, or curvilinear polygoned.

6. The apparatus of claim 1, wherein the photographic image further comprises shading resembling a shadow to indicate the presence of a light source.

7. The apparatus of claim 1, wherein the apparatus is substantially water-proof.

8. The apparatus of claim 1, wherein the apparatus is substantially sweat-proof.

9. The apparatus of claim 1, wherein the first surface is semi-reflective.

10. The apparatus of claim 1, wherein the photographic image depicts nipple accessories.

11. The apparatus of claim 1, wherein the apparatus further comprises a fragrance.

12. The apparatus of claim 1, wherein the apparatus further comprises a flavored coating.

13. An apparatus for realistically simulating the appearance of a nipple-areolar complex, the apparatus comprising:
an adhesive substrate comprising non-woven medical-grade tape for depicting a three-dimensional realistic image of a nipple-areolar complex, and trimmed edges for improving adherence to uneven surfaces, said substrate comprising a first surface and a second surface;
wherein said first surface comprises a lenticular image capable of creating the visual effect of motion and depth of a female human nipple complex, the lenticular image comprising:
a first colored area resembling a human areola, and
a second colored area formed within the first area, wherein the second area resembles a human teat; and
wherein said second flat surface comprising a non-toxic self-adhesive material for adhering said apparatus to human skin.

14. A method for realistically simulating the appearance of a nipple-areolar complex, the method comprising the steps of:
forming an adhesive substrate comprising non-woven medical-grade tape for depicting a realistic image of a nipple-areolar complex and trimmed edges for improving adherence to uneven surfaces, said substrate comprising a first flat surface for printing said image and a second flat surface for adhering to human skin;
creating a photographic image of a nipple-areolar complex, said image comprising a first colored area resembling a human areola, and a second colored area formed within the first area, wherein the second area resembles a human teat;
editing said image of said nipple-areolar complex;
printing said image onto said first flat surface;
applying adhesive to said second flat surface; and
trimming said edges of said substrate to improve the adherence of said substrate onto uneven surface.

15. The method of claim 14, wherein the photographic image depicts the user's own nipple complex.

16. The method of claim 14, wherein the photographic image further comprises computer-generated graphics.

17. The method of claim 14, wherein the photographic image further comprises illustrations.

18. The method of claim 14, wherein the substrate's edges are scalloped, rounded, foiled, or curvilinear polygoned.

19. The method of claim 14, wherein the photographic image further comprises shading resembling a shadow to indicate the presence of a light source.

20. The method of claim 14, wherein the substrate is substantially water-proof.

21. The method of claim 14, wherein the substrate is substantially sweat-proof.

22. The method of claim 14, further comprising the step of applying a semi-reflective coating to the first surface.

23. The method of claim 14, wherein the photographic image depicts nipple accessories.

24. The method of claim 14, further comprising the step of coating the substrate with a fragrance.

25. The method of claim 14, further comprising the step of coating the substrate with a flavoring.
